(19) United States
(12) Patent Application Publication
(10) Pub. No.: US 2025/0336473 A1
(43) Pub. Date: Oct. 30, 2025

(54) METHOD OF BUILDING MODEL FOR MAKING PROGNOSIS OF SURVIVAL RATE OF SUBJECT HAVING BREAST CANCER, AND COMPUTER SYSTEM

(71) Applicant: Pei-Yi CHU, Changhua City (TW)

(72) Inventors: Hung-Yu LIN, Changhua City (TW); Pei-Yi CHU, Changhua City (TW)

(21) Appl. No.: 18/787,788

(22) Filed: Jul. 29, 2024

(30) Foreign Application Priority Data
Apr. 30, 2024 (TW) 113116115

(51) Int. Cl. G16B 25/10; G16B 40/30; G16H 50/30

(52) U.S. Cl. CPC G16B 25/10; G16B 40/30; G16H 50/30

(57) ABSTRACT

A method includes: obtaining pieces of to-be-analyzed data, each of which contains characteristic values related to an age, a stage of breast cancer, a survival condition and gene expression of microRNAs (miRNAs); using univariate analysis based on the pieces of to-be-analyzed data to obtain hazard ratios (HRs) corresponding to the miRNAs and P values corresponding to the HRs; selecting candidate miRNAs from among the miRNAs according to the P values; performing feature selection by using a regression analysis method to select relevant miRNAs from among the candidate miRNAs; using multivariate analysis based on the pieces of to-be-analyzed data to obtain HRs corresponding to the relevant miRNAs and P values corresponding to the HRs; selecting critical miRNAs from among the relevant miRNAs according to the P values; and building a model based on the characteristic values in the pieces of to-be-analyzed data.

### CROSS-REFERENCE TO RELATED APPLICATION

This application claims priority to Taiwanese Invention patent application Ser. No. 11/311,6115, filed on Apr. 30, 2024, and incorporated by reference herein in its entirety.

### FIELD

The disclosure relates to a computer system and a method of building a model for making a prognosis of a survival rate, within a preset time period, of a subject having breast cancer.

### BACKGROUND

A cancer patient is usually eager to know a prognosis of his/her survival rate. Conventionally, values of various biomarkers are used to make a prognosis of a survival rate of a cancer patient. For example, values related to gene expression of different kinds of microRNAs of a breast cancer patient can be used to make a prognosis of his/her survival rate. There are numerous kinds of microRNAs in nature, but not every kind of microRNAs is useful to make a prognosis of a survival rate of a cancer patient.

### SUMMARY

Therefore, an object of the disclosure is to provide a computer system, and a method of building a model for making a prognosis of a survival rate, within a preset time period, of a subject having breast cancer.

According to one aspect of the disclosure, the method includes steps of:
obtaining multiple pieces of to-be-analyzed data that correspond respectively to a plurality of breast-cancer patients, each of the pieces of to-be-analyzed data at least containing a characteristic value related to an age of the corresponding one of the breast-cancer patients, a characteristic value related to a stage of breast cancer in which the corresponding one of the breast-cancer patients is, a characteristic value related to a survival condition of the corresponding one of the breast-cancer patients, and for each of a plurality of microRNAs (miRNAs), a characteristic value corresponding to gene expression of the miRNA for the corresponding one of the breast-cancer patients;
obtaining, by using univariate analysis based on the pieces of to-be-analyzed data, a plurality of hazard ratios (HRs) that correspond respectively to the miRNAs and a plurality of P values that correspond respectively to the HRs corresponding respectively to the miRNAs, each of the HRs that correspond respectively to the miRNAs indicating, for the corresponding one of the miRNAs, a ratio of a survival rate under one gene expression level to a survival rate under another gene expression level;
selecting, according to the P values corresponding respectively to the miRNAs, a plurality of candidate miRNAs from among the miRNAs;
based on the pieces of to-be-analyzed data, performing feature selection by using a regression analysis method to select a plurality of relevant miRNAs from among the candidate miRNAs;
obtaining, by using multivariate analysis based on the pieces of to-be-analyzed data, a plurality of HRs that correspond respectively to the relevant miRNAs and a plurality of P values that correspond respectively to the HRs corresponding respectively to the relevant miRNAs, each of the HRs that correspond respectively to the relevant miRNAs indicating, for the corresponding one of the relevant miRNAs, a ratio of a survival rate under one gene expression level to a survival rate under another gene expression level;
selecting, according to the P values corresponding respectively to the relevant miRNAs, a plurality of critical miRNAs from among the relevant miRNAs;
designating the age, the stage of breast cancer, and the gene expression of at least one of the critical miRNAs as characteristic variables, respectively; and
building the model based on the characteristic values respectively of the characteristic variables in each of the pieces of to-be-analyzed data.

According to another aspect of the disclosure, the computer system is adapted to build a model for making a prognosis of a survival rate, within a preset time period, of a subject having breast cancer. The computer system includes a characteristic-variable designating module and a model-building module.

The characteristic-variable designating module is configured to obtain multiple pieces of to-be-analyzed data that correspond respectively to a plurality of breast-cancer patients. Each of the pieces of to-be-analyzed data at least contains a characteristic value related to an age of the corresponding one of the breast-cancer patients, a characteristic value related to a stage of breast cancer in which the corresponding one of the breast-cancer patients is, a characteristic value related to a survival condition of the corresponding one of the breast-cancer patients, and for each of a plurality of microRNAs (miRNAs), a characteristic value corresponding to gene expression of the miRNA for the corresponding one of the breast-cancer patients.

The characteristic-variable designating module is configured to obtain, by using univariate analysis based on the pieces of to-be-analyzed data, a plurality of hazard ratios (HRs) that correspond respectively to the miRNAs and a plurality of P values that correspond respectively to the HRs corresponding respectively to the miRNAs. Each of the HRs that correspond respectively to the miRNAs indicates, for the corresponding one of the miRNAs, a ratio of a survival rate under one gene expression level to a survival rate under another gene expression level.

The characteristic-variable designating module is configured to select, according to the P values corresponding respectively to the miRNAs, a plurality of candidate miRNAs from among the miRNAs.

The characteristic-variable designating module is configured to, based on the pieces of to-be-analyzed data, perform feature selection by using a regression analysis method to select a plurality of relevant miRNAs from among the candidate miRNAs.

The characteristic-variable designating module is configured to obtain, by using multivariate analysis based on the pieces of to-be-analyzed data, a plurality of HRs that correspond respectively to the relevant miRNAs and a plurality of P values that correspond respectively to the HRs corresponding respectively to the relevant miRNAs. Each of the HRs that correspond respectively to the relevant miRNAs indicates, for the corresponding one of the relevant miRNAs, a ratio of a survival rate under one gene expression level to a survival rate under another gene expression level.

The characteristic-variable designating module is configured to select, according to the P values corresponding respectively to the relevant miRNAs, a plurality of critical miRNAs from among the relevant miRNAs.

The characteristic-variable designating module is configured to designate the age, the stage of breast cancer, and the gene expression of at least one of the critical miRNAs as characteristic variables, respectively.

The model-building module is configured to build the model based on the characteristic values respectively of the characteristic variables in each of the pieces of to-be-analyzed data.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating a computer system according to an embodiment of the disclosure.

FIG. 2 is a flow chart illustrating a method of building a model for making a prognosis of a survival rate, within a preset time period, of a subject having breast cancer according to an embodiment of the disclosure.

FIG. 3 is a schematic diagram illustrating an example of the model that is expressed in a form of a nomogram according to an embodiment of the disclosure.

FIG. 4 is a schematic diagram illustrating an example of receiver operating characteristic (ROC) curves for prognoses made by a model that is built based on characteristic values related to an age, a stage of breast cancer, and gene expression of miR-342 according to an embodiment of the disclosure.

FIG. 5 is a schematic diagram illustrating an example of ROC curves for prognoses made by a model that is built based on characteristic values related to an age, a stage of breast cancer, and gene expression of miR-340 according to an embodiment of the disclosure.

FIG. 6 is a schematic diagram illustrating an example of ROC curves for prognoses made by a model that is built based on characteristic values related to an age, a stage of breast cancer, and gene expression of miR-133a according to an embodiment of the disclosure.

FIG. 7 is a schematic diagram illustrating an example of ROC curves for prognoses made by a model that is built based on characteristic values related to an age, a stage of breast cancer, and gene expression of miR-128 according to an embodiment of the disclosure.

FIG. 8 is a schematic diagram illustrating an example of ROC curves for prognoses made by a model that is built based on characteristic values related to an age, a stage of breast cancer, and gene expression of let-7a according to an embodiment of the disclosure.

FIG. 9 is a schematic diagram illustrating an example of ROC curves for prognoses made by a model that is built based on characteristic values related to an age, a stage of breast cancer, and gene expression of miR-342, miR-340, miR-133a, miR-128 and let-7a according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

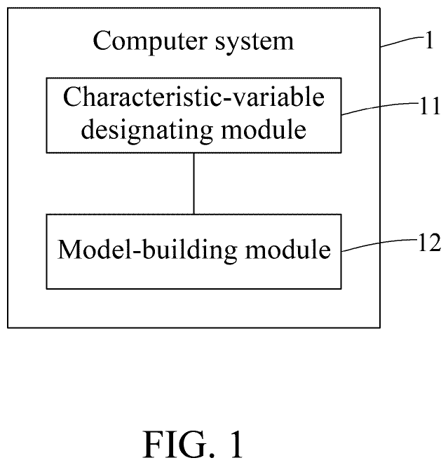

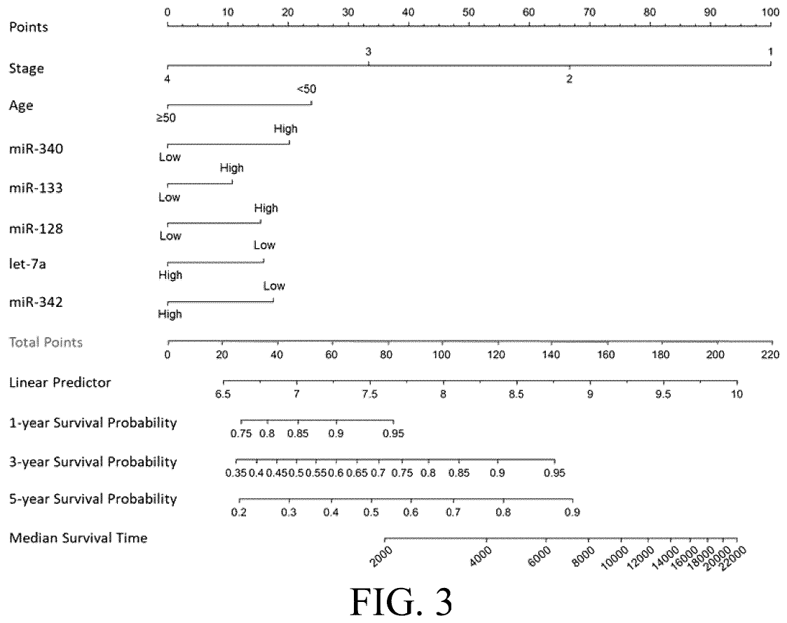

Referring to FIG. 1, a computer system 1 according to an embodiment of the disclosure is illustrated. The computer system 1 is adapted to build a model for making a prognosis of a survival rate, within a preset time period (e.g., one year, three years, five years, etc.), of a subject (e.g., a human being) having breast cancer. The computer system 1 may be implemented to be a desktop computer, a laptop computer, a notebook computer or a tablet computer, but implementation thereof is not limited to what are disclosed herein and may vary in other embodiments. In particular, the model is expressed in a form of a nomogram (see FIG. 3).

The computer system 1 includes a characteristic-variable designating module 11 and a model-building module 12 that are electrically connected to each other.

It should be noted that each of the characteristic-variable designating module 11 and the model-building module 12 may be implemented by one of hardware, firmware, software, and any combination thereof. For example, the characteristic-variable designating module 11 and the model-building module 12 may be implemented to be software modules in a program, where the software modules contain codes and instructions to carry out specific functionalities, and can be called individually or together to fulfill the computer system 1 of this disclosure.

The above-mentioned modules may be embodied in: executable software as a set of logic instructions stored in a machine-or computer-readable storage medium of a memory such as random access memory (RAM), read only memory (ROM), programmable ROM (PROM), firmware, flash memory, etc.; configurable logic such as programmable logic arrays (PLAs), field programmable gate arrays (FPGAs), complex programmable logic devices (CPLDs), etc.; fixed-functionality logic hardware using circuit technology such as application specific integrated circuit (ASIC), complementary metal oxide semiconductor (CMOS), transistor-transistor logic (TTL) technology, etc.; or any combination thereof.

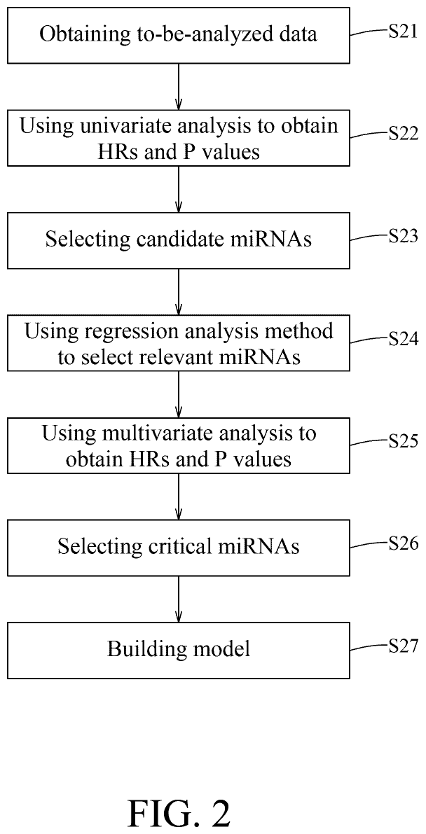

Referring to FIG. 2, a method of building the model according to an embodiment of the disclosure is illustrated. The method is to be implemented by the computer system 1 that is previously described. The method includes steps S21 to S27 delineated below.

In step S21, the characteristic-variable designating module 11 is configured to obtain multiple pieces of to-be-analyzed data that correspond respectively to a plurality of breast-cancer patients. Each of the pieces of to-be-analyzed data at least contains a characteristic value related to an age of the corresponding one of the breast-cancer patients, a characteristic value related to a stage of breast cancer in which the corresponding one of the breast-cancer patients is, a characteristic value related to a survival condition of the corresponding one of the breast-cancer patients, a characteristic value related to a follow-up time for the corresponding one of the breast-cancer patients, and for each of a plurality of microRNAs (miRNAs), a characteristic value corresponding to gene expression of the miRNA for the corresponding one of the breast-cancer patients. In this embodiment, the pieces of to-be-analyzed data are obtained from the Cancer Genome Atlas Breast Invasive Carcinoma (TCGA-BRCA) project. In addition, there are about thirty kinds of miRNAs used in this embodiment, and all miRNAs used in this embodiment are related to mitochondrial metabolism.

In step S22, the characteristic-variable designating module 11 is configured to obtain, by using univariate analysis based on the pieces of to-be-analyzed data, a plurality of hazard ratios (HRs) that correspond respectively to the miRNAs and a plurality of P values that correspond respectively to the HRs corresponding respectively to the miRNAs. In this embodiment, the univariate analysis is implemented by using analysis tools provided by the Hiplot visualization platform. Each of the HRs that correspond respectively to the miRNAs indicates, for the corresponding one of the miRNAs, a ratio of a survival rate under one gene expression level to a survival rate under another gene expression level. For one of the miRNAs, the survival rate under one gene expression level is a proportion of a number of a group of the breast-cancer patients to a total number of the breast-cancer patients, wherein for each one in the group of the breast-cancer patients, the characteristic value related to the survival condition indicates a condition of being alive and the characteristic value related to the gene expression of the miRNA is at the one gene expression level.

In this embodiment, each of the HRs corresponding respectively to the miRNAs indicates, for the corresponding one of the miRNAs, a ratio of a survival rate under a high gene expression level to a survival rate under a low gene expression level. Specifically, for one of the miRNAs, the high gene expression level is a gene expression level of the miRNA not less than a preset threshold, and the low gene expression level is a gene expression level of the miRNA less than the preset threshold. For one of the miRNAs, the survival rate under the high gene expression level is a proportion of a number of a group of the breast-cancer patients to a total number of the breast-cancer patients, wherein for each one in the group of the breast-cancer patients, the characteristic value related to the survival condition indicates a condition of being alive and the characteristic value related to the gene expression of the miRNA is at the high gene expression level. Similarly, for one of the miRNAs, the survival rate under the low gene expression level is a proportion of a number of another group of the breast-cancer patients to the total number of the breast-cancer patients, wherein for each one in the another group of the breast-cancer patients, the characteristic value related to the survival condition indicates a condition of being alive and the characteristic value related to the gene expression of the miRNA is at the low gene expression level.

In step S23, the characteristic-variable designating module 11 is configured to select, according to the P values corresponding respectively to the miRNAs, a plurality of candidate miRNAs from among the miRNAs. Specifically, the candidate miRNAs selected by the characteristic-variable designating module 11 are those of the miRNAs to which the P values respectively correspond and which are less than 0.05. It is worth to note that for each of the candidate miRNAs, the P value corresponding to the candidate miRNA that is less than 0.05 stands for a significant difference between survival rates under a high gene expression level and a low gene expression level of the candidate miRNA. In this embodiment, twelve candidate miRNAs were selected, and the twelve candidate miRNAs are miR-340, miR-133a, miR-128, let-7a, miR-29c, miR-223, miR-342, miR-26a, miR-29a, miR-150, miR-195 and miR-146a. In this way, a number of the characteristic values related to the miRNAs that will be further utilized to build the model may be reduced.

In step S24, the characteristic-variable designating module 11 is configured to, based on the pieces of to-be-analyzed data, perform feature selection by using a regression analysis method to select a plurality of relevant miRNAs from among the candidate miRNAs. In this embodiment, the regression analysis method is least absolute shrinkage and selection operator (LASSO). In LASSO, each of the candidate miRNAs will be assigned with a coefficient that is to be multiplied with the characteristic value corresponding to gene expression of said each of the candidate miRNAs. Table 1 below shows the candidate miRNAs and the coefficients assigned respectively to the candidate miRNAs. Only those of the candidate miRNAs assigned with non-zero coefficients are reserved as the relevant miRNAs. That is to say, as shown in Table 1, in this embodiment, the characteristic-variable designating module 11 selects eight relevant miRNAs: miR-146a, miR-195,miR-128, miR-342, miR-29c, let-7a, miR-133 and miR-340.

TABLE 1

| Candidate miRNA | Coefficient |
|---|---|
| miR-146a | −0.207821956 |
| miR-195 | −0.089080422 |
| miR-150 | 0 |
| miR-26a | 0 |
| miR-342 | −0.065340221 |
| miR-223 | 0 |
| miR-29c | −0.00449019 |
| let-7a | −0.178840671 |
| miR-128 | 0.124470123 |
| miR-133a | 0.068987395 |
| miR-340 | 0.208628989 |
| miR-29a | 0 |

In step S25, the characteristic-variable designating module 11 is configured to obtain, by using multivariate analysis based on the pieces of to-be-analyzed data, a plurality of HRs that correspond respectively to the relevant miRNAs and a plurality of P values that correspond respectively to the HRs corresponding respectively to the relevant miRNAs. In this embodiment, the multivariate analysis is implemented by using analysis tools provided by the Hiplot visualization platform. Each of the HRs that correspond respectively to the relevant miRNAs indicates, for the corresponding one of the relevant miRNAs, a ratio of a survival rate under one gene expression level to a survival rate under another gene expression level. Particularly, each of the HRs corresponding respectively to the relevant miRNAs indicates, for the corresponding one of the relevant miRNAs, a ratio of a survival rate under a high gene expression level to a survival rate under a low gene expression level. Since definitions of the survival rate under the high gene expression level and the survival rate under the low gene expression level for the relevant miRNAs are similar to definitions of the survival rate under the high gene expression level and the survival rate under the low gene expression level for the miRNAs, a repeated explanation is omitted herein for the sake of brevity.

In step S26, the characteristic-variable designating module 11 is configured to select, according to the P values corresponding respectively to the relevant miRNAs, a plurality of critical miRNAs from among the relevant miRNAs. Specifically, the critical miRNAs selected by the characteristic-variable designating module 11 are those of the relevant miRNAs to which the P values respectively correspond and which are less than 0.05. In this embodiment, the critical miRNAs eventually selected by the characteristic-variable designating module 11 are miR-342, miR-340, miR-133a, miR-128 and let-7a.

In step S27, the characteristic-variable designating module 11 is configured to designate the age, the stage of breast cancer, and the gene expression of at least one of the critical miRNAs as characteristic variables, respectively. Then, the model-building module 12 is configured to build the model based on the characteristic values respectively of the characteristic variables in each of the pieces of to-be-analyzed data. The model, which is expressed as the nomogram, as illustrated in FIG. 3 is built based on the characteristic values of the age, the stage of breast cancer, and the gene expression of the five critical miRNAs (i.e., miR-342, miR-340, miR-133a, miR-128 and let-7a). The model can be utilized to make a prognosis of a survival rate, within one year, three years, or five years, of a subject having breast cancer. Since implementation of building the nomogram has been well known to one skilled in the relevant art, detailed explanation of the same is omitted herein for the sake of brevity.

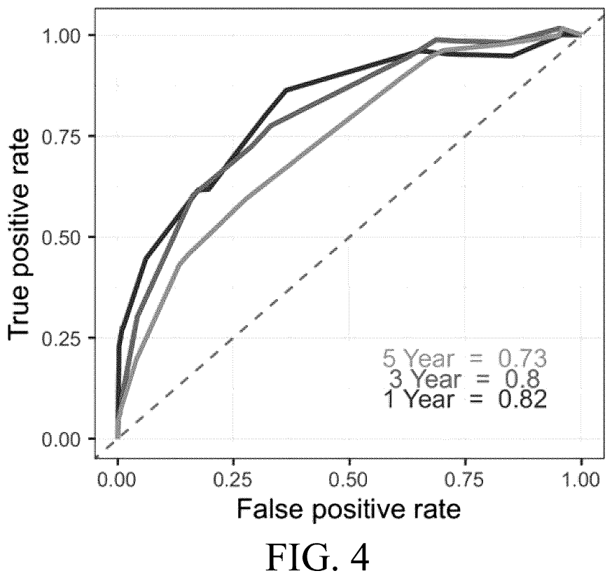

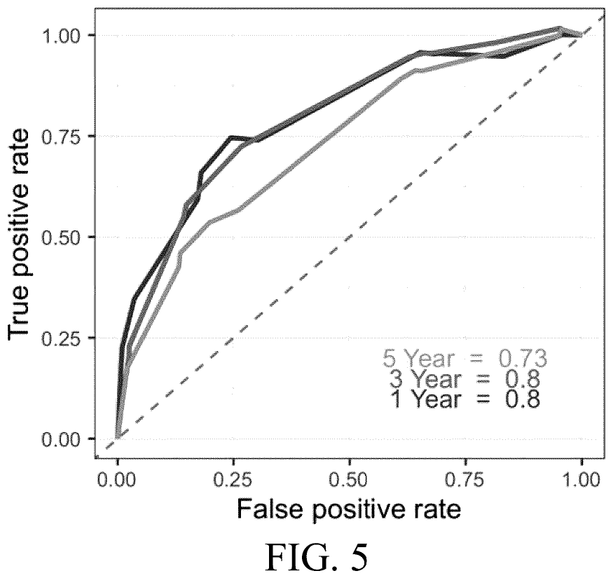

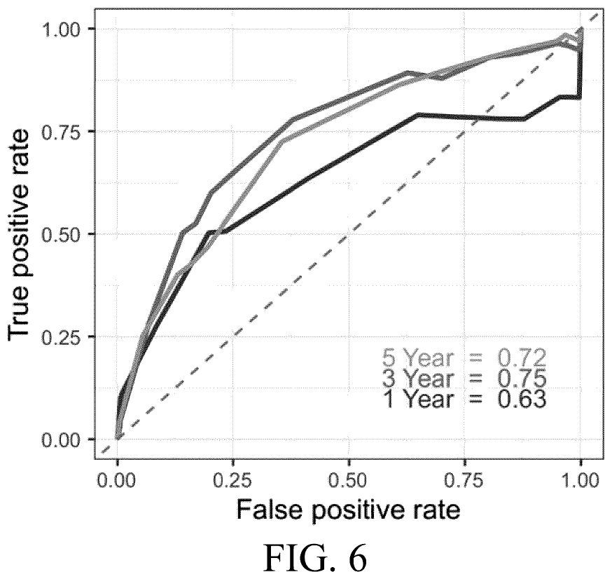

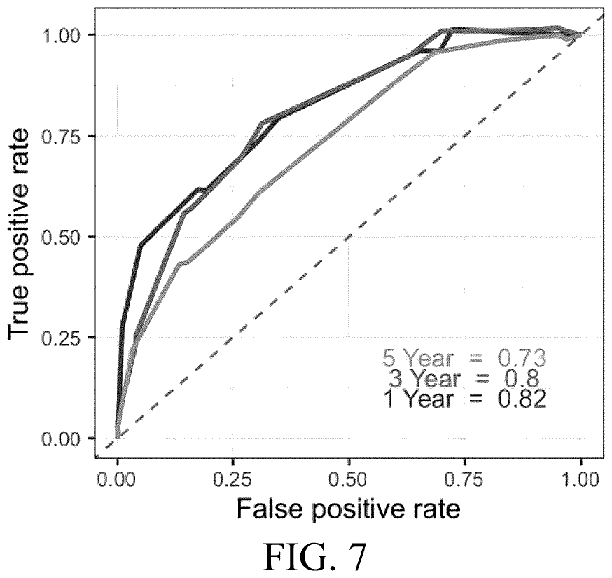

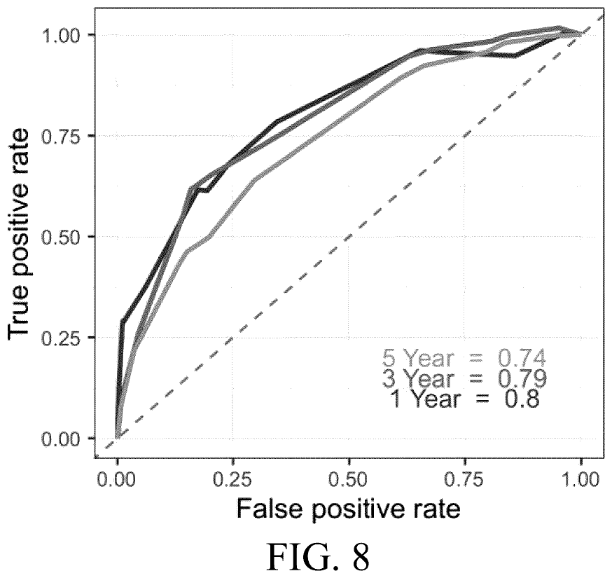

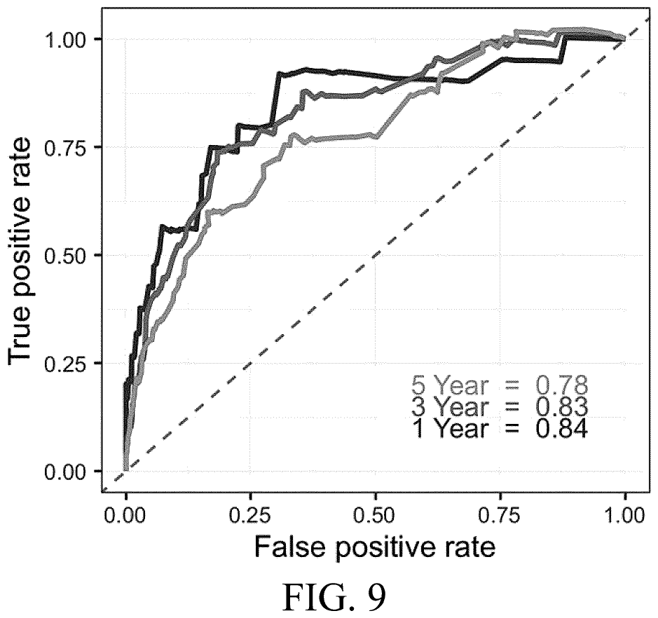

Each of FIGS. 4 to 9 illustrates three receiver operating characteristic (ROC) curves for making prognoses of a survival rate respectively within one year, three years and five years by using the model built in steps S21 to S27. The prognoses in FIG. 9 are made by using the model built based on the characteristic values related to the age, the stage of breast cancer, and the gene expression of all of miR-342, miR-340, miR-133a, miR-128 and let-7a. Comparatively, the prognoses in FIGS. 4 to 8 are made by using the model built based on the characteristic values related to the age, the stage of breast cancer, and the gene expression respectively of miR-342 (see FIG. 4), miR-340 (see FIG. 5), miR-133a (see FIG. 6), miR-128 (see FIG. 7) and let-7a (see FIG. 8). It is worth to note that for each of one year, three years and five years, an area under curve (AUC) of the corresponding one of the three ROC curves in FIG. 9 is less than an AUC of the corresponding one of the three ROC curves in each of FIGS. 4 to 8. Therefore, a prognosis made by using the model built based on the characteristic values related to the age, the stage of breast cancer, and the gene expression of all of miR-342, miR-340, miR-133a, miR-128 and let-7a may achieve relatively higher accuracy.

To sum up, for the computer system 1 and the method of building a model for making a prognosis of a survival rate, within a preset time period, of a subject having breast cancer according to the disclosure, the univariate analysis, the regression analysis method and the multivariate analysis are sequentially used to select the critical miRNAs, which are the most critical in making the aforesaid prognosis, and the model is built based on characteristic values related to an age, a stage of breast cancer, and gene expression of at least one of the critical miRNAs contained in each of the pieces of to-be-analyzed data. In this way, accuracy of prognosis made by using the model may be relatively high.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method of building a model for making a prognosis of a survival rate, within a preset time period, of a subject having breast cancer, the method comprising:
obtaining multiple pieces of to-be-analyzed data that correspond respectively to a plurality of breast-cancer patients, each of the pieces of to-be-analyzed data at least containing a characteristic value related to an age of the corresponding one of the breast-cancer patients, a characteristic value related to a stage of breast cancer in which the corresponding one of the breast-cancer patients is, a characteristic value related to a survival condition of the corresponding one of the breast-cancer patients, and for each of a plurality of microRNAs (miRNAs), a characteristic value corresponding to gene expression of the miRNA for the corresponding one of the breast-cancer patients;
obtaining, by using univariate analysis based on the pieces of to-be-analyzed data, a plurality of hazard ratios (HRs) that correspond respectively to the miRNAs and a plurality of P values that correspond respectively to the HRs corresponding respectively to the miRNAs, each of the HRs that correspond respectively to the miRNAs indicating, for the corresponding one of the miRNAs, a ratio of a survival rate under one gene expression level to a survival rate under another gene expression level;
selecting, according to the P values corresponding respectively to the miRNAs, a plurality of candidate miRNAs from among the miRNAs;
based on the pieces of to-be-analyzed data, performing feature selection by using a regression analysis method to select a plurality of relevant miRNAs from among the candidate miRNAs;
obtaining, by using multivariate analysis based on the pieces of to-be-analyzed data, a plurality of HRs that correspond respectively to the relevant miRNAs and a plurality of P values that correspond respectively to the HRs corresponding respectively to the relevant miRNAs, each of the HRs that correspond respectively to the relevant miRNAs indicating, for the corresponding one of the relevant miRNAs, a ratio of a survival rate under one gene expression level to a survival rate under another gene expression level;
selecting, according to the P values corresponding respectively to the relevant miRNAs, a plurality of critical miRNAs from among the relevant miRNAs;
designating the age, the stage of breast cancer, and the gene expression of at least one of the critical miRNAs as characteristic variables, respectively; and
building the model based on the characteristic values respectively of the characteristic variables in each of the pieces of to-be-analyzed data.

2. The method as claimed in claim 1, wherein:
each of the HRs corresponding respectively to the miRNAs indicates, for the corresponding one of the miRNAs, a ratio of a survival rate under a high gene expression level to a survival rate under a low gene expression level; and
each of the HRs corresponding respectively to the relevant miRNAs indicates, for the corresponding one of the relevant miRNAs, a ratio of a survival rate under a high gene expression level to a survival rate under a low gene expression level.

3. The method as claimed in claim 2, wherein for one of the miRNAs, the high gene expression level is a gene expression level of the miRNA not less than a preset threshold, and the low gene expression level is a gene expression level of the miRNA less than the preset threshold.

4. The method as claimed in claim 3, wherein for one of the miRNAs:
the survival rate under the high gene expression level is a proportion of a number of a group of the breast-cancer patients to a total number of the breast-cancer patients, for each one in the group of the breast-cancer patients, the characteristic value related to the survival condition indicates a condition of being alive and the characteristic value related to the gene expression of the miRNA is at the high gene expression level; and
the survival rate under the low gene expression level is a proportion of a number of another group of the breast-cancer patients to the total number of the breast-cancer patients, for each one in the another group of the breast-cancer patients, the characteristic value related to the survival condition indicates a condition of being alive and the characteristic value related to the gene expression of the miRNA is at the low gene expression level.

5. The method as claimed in claim 1, wherein the critical miRNAs are miR-342, miR-340, miR-133a, miR-128 and let-7a.

6. The method as claimed in claim 1, wherein the regression analysis method is least absolute shrinkage and selection operator (LASSO).

7. The method as claimed in claim 1, wherein the model is expressed in a form of a nomogram.

8. The method as claimed in claim 1, wherein for one of the miRNAs, the survival rate under one gene expression level is a proportion of a number of a group of the breast-cancer patients to a total number of the breast-cancer patients, for each one in the group of the breast-cancer patients, the characteristic value related to the survival condition indicates a condition of being alive and the characteristic value related to the gene expression of the miRNA is at the one gene expression level.

9. A computer system adapted to build a model for making a prognosis of a survival rate, within a preset time period, of a subject having breast cancer, comprising:
a characteristic-variable designating module configured to
obtain multiple pieces of to-be-analyzed data that correspond respectively to a plurality of breast-cancer patients, each of the pieces of to-be-analyzed data at least containing a characteristic value related to an age of the corresponding one of the breast-cancer patients, a characteristic value related to a stage of breast cancer in which the corresponding one of the breast-cancer patients is, a characteristic value related to a survival condition of the corresponding one of the breast-cancer patients, and for each of a plurality of microRNAs (miRNAs), a characteristic value corresponding to gene expression of the miRNA for the corresponding one of the breast-cancer patients,
obtain, by using univariate analysis based on the pieces of to-be-analyzed data, a plurality of hazard ratios (HRs) that correspond respectively to the miRNAs and a plurality of P values that correspond respectively to the HRs corresponding respectively to the miRNAs, each of the HRs that correspond respectively to the miRNAs indicating, for the corresponding one of the miRNAs, a ratio of a survival rate under one gene expression level to a survival rate under another gene expression level,
select, according to the P values corresponding respectively to the miRNAs, a plurality of candidate miRNAs from among the miRNAs,
based on the pieces of to-be-analyzed data, perform feature selection by using a regression analysis method to select a plurality of relevant miRNAs from among the candidate miRNAs,
obtain, by using multivariate analysis based on the pieces of to-be-analyzed data, a plurality of HRs that correspond respectively to the relevant miRNAs and a plurality of P values that correspond respectively to the HRs corresponding respectively to the relevant miRNAs, each of the HRs that correspond respectively to the relevant miRNAs indicating, for the corresponding one of the relevant miRNAs, a ratio of a survival rate under one gene expression level to a survival rate under another gene expression level,
select, according to the P values corresponding respectively to the relevant miRNAs, a plurality of critical miRNAs from among the relevant miRNAs, and
designate the age, the stage of breast cancer, and the gene expression of at least one of the critical miRNAs as characteristic variables, respectively; and
a model-building module configured to build the model based on the characteristic values respectively of the characteristic variables in each of the pieces of to-be-analyzed data.

10. The computer system as claimed in claim 9, wherein:
each of the HRs corresponding respectively to the miRNAs indicates, for the corresponding one of the miRNAs, a ratio of a survival rate under a high gene expression level to a survival rate under a low gene expression level; and
each of the HRs corresponding respectively to the relevant miRNAs indicates, for the corresponding one of the relevant miRNAs, a ratio of a survival rate under a high gene expression level to a survival rate under a low gene expression level.

11. The computer system as claimed in claim 10, wherein for one of the miRNAs, the high gene expression level is a gene expression level of the miRNA not less than a preset threshold, and the low gene expression level is a gene expression level of the miRNA less than the preset threshold.

12. The computer system as claimed in claim 11, wherein for one of the miRNAs:
the survival rate under the high gene expression level is a proportion of a number of a group of the breast-cancer patients to a total number of the breast-cancer patients, for each one in the group of the breast-cancer patients, the characteristic value related to the survival condition indicates a condition of being alive and the characteristic value related to the gene expression of the miRNA is at the high gene expression level; and
the survival rate under the low gene expression level is a proportion of a number of another group of the breast-cancer patients to the total number of the breast-cancer patients, for each one in the another group of the breast-cancer patients, the characteristic value related to the survival condition indicates a condition of being alive and the characteristic value related to the gene expression of the miRNA is at the low gene expression level.

13. The computer system as claimed in claim 9, wherein the critical miRNAs are miR-342, miR-340, miR-133a, miR-128 and let-7a.

14. The computer system as claimed in claim 9, wherein the regression analysis method is least absolute shrinkage and selection operator (LASSO).

15. The computer system as claimed in claim 9, wherein the model is expressed in a form of a nomogram.

16. The computer system as claimed in claim 9, wherein for one of the miRNAs, the survival rate under one gene expression level is a proportion of a number of a group of the breast-cancer patients to a total number of the breast-cancer patients, for each one in the group of the breast-cancer patients, the characteristic value related to the survival condition indicates a condition of being alive and the characteristic value related to the gene expression of the miRNA is at the one gene expression level.
